(19) United States
(12) Patent Application Publication
(10) Pub. No.: US 2023/0218611 A1
(43) Pub. Date: Jul. 13, 2023

(54) OPHTHALMIC COMPOSITIONS COMPRISING A COMBINATION OF BRINZOLAMIDE AND BRIMONIDINE

(71) Applicant: SENTISS PHARMA PRIVATE LIMITED, New Delhi (IN)

(72) Inventors: Manoj NAHAR, GURUGRAM, HARYANA (IN); Shailendra PRASAD, GURUGRAM, HARYANA (IN); Mallinath HARWALKAR, GURUGRAM, HARYANA (IN); Kishor DEO, GURUGRAM, HARYANA (IN); Deepak BAHRI, GURUGRAM, HARYANA (IN)

(21) Appl. No.: 17/997,203

(22) PCT Filed: Apr. 28, 2021
(86) PCT No.: PCT/IB2021/053533

§ 371 Date: Oct. 26, 2022

(30) Foreign Application Priority Data
Apr. 29, 2020 (IN) 202011018367

(51) Int. Cl. A61K 31/498; A61K 31/542; A61K 47/36; A61K 47/10; A61K 9/00; A61P 27/06

(52) U.S. Cl. CPC A61K 31/498; A61K 31/542; A61K 47/36; A61K 47/10; A61K 9/0048; A61P 27/06

(57) ABSTRACT

An aqueous ophthalmic composition, comprising: a therapeutically effective amount of brimonidine or their salts thereof; a therapeutically effective amount of brinzolamide or their salts thereof; buffers at a concentration that is at least about 0.05% w/v to 5.0% w/v of the ophthalmic composition; a preservative and; pharmaceutically acceptable excipients, wherein the pH of said composition is less than 8.0.

### TECHNICAL FIELD OF THE INVENTION

The present invention is directed to the provision of multi-dose, aqueous pharmaceutical compositions comprising two or more therapeutic agents selected from brinzolamide, brimonidine or a combination thereof in conjunction with a preservative other than benzalkonium chloride (BAC). Instead, the compositions contain either Benzododecinium Bromide (BDDB) or Sodium Perborate or Polyquaternium-1 as a preservative for improved preservation of the compositions.

More particularly, the present invention is related to multi-dose, pharmaceutical ophthalmic compositions that lacks borate-polyol complex formation and without use of BAC even at low concentration and still possess sufficient antimicrobial activity to satisfy USP preservative efficacy requirements, as well as similar other preservative standards.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. In particular, a glaucoma patient will develop peripheral visual field loss followed by central field loss usually in the presence of elevated intraocular pressure, which if left untreated it may eventually lead to blindness.

Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma. Most patients with glaucoma are treated with topical medication that controls elevated ocular pressure. Medications most commonly used are Alpha (α)-adrenergic receptor agonists, epinephrine compounds, prostaglandins that reduce ocular pressure by increasing aqueous outflow, β-adrenergic receptor antagonists and carbonic anhydrase inhibitors that work by decreasing aqueous production. Even though the typical treatment regimen for lowering intraocular pressure is topical β-blockers, in the recent years the use of prostaglandins as initial therapy is increased.

Carbonic anhydrase inhibitors are also used for the treatment of ocular hypertension related to glaucoma. The drugs that belong to this family inhibit the enzyme carbonic anhydrase and thus, they reduce the contribution of the aqueous humor formation made by the carbonic anhydrase pathway. However, these drugs cannot be used via a systemic route because they would inhibit the enzymatic activity of carbonic anhydrase throughout the entire body. In general, the enzyme carbonic anhydrase plays a major role in regulating pH and fluid levels in the human body by converting carbon dioxide to carbonic acid and bicarbonate ions.

Brinzolamide R-(+)-4ethylamino-3,4-dihydro-2-(3-methoxy)propyl-2H thieno[3,2,e]1,2-thiazene-6 sulfonamide-1,1-dioxide) is a carbonic anhydrase inhibitor disclosed in U.S. Pat. No. 5,378,703 and sold in a topical ophthalmic formulation (Azopt™) for lowering elevated intra-ocular pressure (IOP) in patients with open-angle glaucoma or ocular hypertension (OHT) (Alcon Laboratories, Inc., Fort Worth, Tex.).

Alpha-2-adrenergic agonist is another well characterized class of drugs used for the treatment of high intraocular pressure related with glaucoma. The drugs of this class act via decreasing synthesis of aqueous humor and increasing the amount that drains from the eye through uveoscleral outflow.

The present invention relates to compositions containing alpha-2-adrenergic agonists. Brimonidine tartrate ((5-bromo-6-2-imidzolidisnylideneamino) quinozoline L-tartrate) hereinafter “brimonidine” is a relatively selective alpha-2-adrenergic agonist sold in a topical ophthalmic formulation (Alphagan™) for lowering elevated IOP in patients with open angle-glaucoma or ocular hypertension (Allergan, Inc., Irvine, Calif.).

The topical use of brimonidine to lower intraocular pressure in patients with glaucoma or ocular hypertension is known. The first ophthalmic brimonidine product in the U.S. was approved by the FDA in 1996. That product, sold under the trade name Alphagan, contained brimonidine in the form of brimonidine tartrate at a concentration of 0.2%. The preservative contained in Alphagan is benzalkonium chloride, the most widely used preservative for topical ophthalmic compositions.

In 2001, a second ophthalmic brimonidine product was approved by the U.S. FDA. This product, sold under the trade name Alphagan® P, contained brimonidine tartrate at two brimonidine concentrations, 0.15% and 0.1%, each of which is lower than the 0.2% brimonidine concentration in Alphagan®. Alphagan® P has a pH range that is higher than that of Alphagan®. According to the product label, the lower concentration Alphagan® P formulation is sold at a pH of 7.4 to 8.0; the higher concentration is sold at a pH of 6.6 to 7.4. The preservative contained in Alphagan® P is chlorine dioxide. See U.S. Pat. Nos. 5,424,078 and 6,562,873.

U.S. Pat. No. 6,316,441 discloses the use of brinzolamide in combination with brimonidine to treat ocular diseases which have their etiology in compromised blood flow.

These diseases include, but are not limited to glaucoma, occlusion conditions, diabetic retinopathy, and ocular neovascularization. These agents can be used either alone, in separate compositions dosed within 5 to 10 min of each other, or together in a single formulation. In all the formulations, it discloses the use of Benzalkonium Chloride as a preservative.

The U.S. Pat. Nos. 9,421,265 and 9,044,484 discloses pharmaceutical compositions that contain borate-polyol complexes for improved preservation of the compositions. More specifically the present invention relates to aqueous pharmaceutical compositions (e.g., multi-dose ophthalmic compositions) containing two or more different polyols in conjunction with borate and a preservative, particularly benzalkonium chloride (BAC).

Further, it is well established in the prior that a detergent preservative used in the above references such as benzalkonium chloride was known to be somewhat irritating to the eye. It is well known in the reference literature that small organic compounds, such as benzalkonium chloride (BAC), chlorhexidine, thimerosal have excellent antimicrobial activity; however, it is now known that these small organic antimicrobials are often toxic to the sensitive tissues of the eye and can accumulate in cornea, contact lenses, particularly soft, hydrophilic contact lenses. Medications with BAC may cause disruption of the corneal surface with lower concentrations of BAC.

Gasset and Grant et al. showed that BAC accumulates in ocular tissue and remains there for long periods, adversely affecting both the corneal surface and the conjunctiva. Therefore, cessation of the medications may not immediately improve the condition and function of the ocular surface. These findings also suggest that corneal cell necrosis may occur in some patients who are taking multiple BAC-preserved ocular medications over long periods of time, even when the amount of BAC in any one medication is below the threshold concentration at which necrosis occurs.

The U.S. Pat. Nos. 9,421,265 and 9,044,484 further disclose that it would be particularly desirable to provide an ophthalmic composition, which includes borate-polyol complex formed with lower concentrations of particular polyols and/or borate and includes low concentrations of BAC while exhibiting improved anti-microbial activity and desirable buffering activity. However, in the present invention, the inventors have surprisingly and unexpectedly found that their formulations are stable and achieved the preservative efficacy without having any borate-polyol complexes therefore there is no need of these excipients in their formulation.

So, there is an unmet medical need to prepare a pharmaceutical composition comprising brinzolamide or their salts thereof, brimonidine or their salts thereof or a combination thereof that contain preservatives other than benzalkonium chloride (BAC), the other preservatives being less toxic than BAC in nature. Instead, the compositions contain either Benzododecinium Bromide (BDDB) or Sodium Perborate or Polyquaternium-1 as a preservative for improved preservation of the composition, which is less irritating to the eye(s) of a patient in need thereof.

Therefore, the present invention is directed to pharmaceutical compositions comprising a combination of brinzolamide and brimonidine or their salts thereof that contain preservatives other than benzalkonium chloride (BAC). Preferably, the compositions contain Benzododecinium Bromide (BDDB) as a preservative for improved preservation of the compositions.

### SUMMARY OF THE INVENTION

The present invention is directed to a multi-dose, ophthalmic compositions comprising Brinzolamide or their pharmaceutically acceptable salts thereof and Brimonidine or their pharmaceutically acceptable salts thereof, in combination with a preservative(s) other than benzalkonium chloride (BAC). Instead, the ophthalmic compositions contain either Benzododecinium Bromide (BDDB) or Sodium Perborate or Polyquaternium-1 as a preservative for improved preservation of the composition(s).

Preferably, the ophthalmic compositions contain Benzododecinium Bromide (BDDB) as a sole preservative to preserve the composition(s) of the present invention.

Preferably, the ophthalmic compositions contain Sodium Perborate as a sole preservative to preserve the composition(s) of the present invention.

Preferably, the ophthalmic compositions contain Polyquaternium-1 as a sole preservative to preserve the composition(s) of the present invention.

Particularly, the present invention is directed to topical ophthalmic compositions for the decrease of intraocular pressure (IOP) in patients with ocular hypertension or open angle glaucoma comprising a combination of Brinzolamide and Brimonidine or their pharmaceutical acceptable salts thereof optionally with pharmaceutically acceptable excipients and a process for the preparation thereof.

More particularly, the present invention is directed to develop a stable ophthalmic composition that lacks borate-polyol complex formation and is free of microbes during storage and for the duration of use; such formulation would provide a significant improvement in preservation over the prior art formulations.

### BRIEF DESCRIPTION OF THE DRAWINGS

FIG. 1 Comparison of Zeta Potential (mV) of reference products and present invention formulations 5a, 5b & 5c.

FIG. 2 Comparison of Surface Tension (mN/m) of reference products and present invention formulations 5a, 5b & 5c.

FIG. 3 Drug release profile of reference products and present invention formulations 5a, 5b & 5c.

FIG. 4 Comparison of 2 theta value of reference products and present invention formulations 5a, 5b & 5c.

### DETAILED DESCRIPTION OF THE INVENTION

The present invention is directed to a multi-dose, aqueous pharmaceutical compositions comprising two or more therapeutic agents selected from brinzolamide or their salts thereof, brimonidine or their salts thereof or a combination thereof, along with a preservative other than benzalkonium chloride (BAC).

Instead, the compositions contain either Benzododecinium Bromide (BDDB) or Sodium Perborate or Polyquaternium-1 as a preservative for improved preservation of the present composition(s).

Preferably, the ophthalmic compositions contain Benzododecinium Bromide (BDDB) as a sole preservative to preserve the composition of the present invention.

Preferably, the ophthalmic compositions contain Sodium Perborate as a sole preservative to preserve the composition(s) of the present invention.

Preferably, the ophthalmic compositions contain Polyquaternium-1 as a sole preservative to preserve the composition(s) of the present invention.

More particularly, the present invention is related to multi-dose, pharmaceutical ophthalmic compositions that lacks borate-polyol complex formation and still possess sufficient antimicrobial activity to satisfy USP preservative efficacy requirements, as well as similar other preservative standards.

As used herein, the term “BAC/BKC” wherever appears in the specification is an abbreviation for “benzalkonium chloride”.

As used herein, the “BDDB” wherever appears in the specification is an abbreviation for “Benzododecinium Bromide”.

As used herein, the “NMT” wherever appears is an abbreviation for “not more than” and the “RH” wherever appears is an abbreviation for “relative humidity”.

As used herein, the “IOP” wherever appears is an abbreviation for “intraocular pressure”.

Unless indicated otherwise, all ingredient amounts are presented in units of % weight/volume (% w/v).

Brimonidine tartrate is a known compound that can be made by known methods and is commercially available. See, for example, German Patent No. 2,538,620.

In one embodiment, the present invention is directed to a multi-dose, ophthalmic compositions comprising a combination of Carbonic anhydrase inhibitors or their pharmaceutically acceptable salts thereof and alpha-2-adrenergic agonist or their pharmaceutically acceptable salts thereof, optionally with other pharmaceutical acceptable excipients.

In another embodiment, the present invention is directed to an aqueous ophthalmic composition comprising a combination of Carbonic anhydrase inhibitors and alpha-2-adrenergic agonist in combination with a preservative other than benzalkonium chloride (BAC).

Instead, the compositions contain either Benzododecinium Bromide (BDDB) or Sodium Perborate or Polyquaternium-1 as a preservative for the improved preservation of the compositions of the present invention.

In one embodiment, the ophthalmic compositions contain Sodium Perborate as a sole preservative to preserve the composition of the present invention.

In another embodiment, the ophthalmic compositions contain Polyquaternium-1 as a sole preservative to preserve the composition of the present invention.

According to the present invention, a carbonic anhydrase inhibitor is selected from Brinzolamide or a pharmaceutically acceptable salt or solvate or hydrate thereof wherein the concentration of brinzolamide in the composition is from about 0.01-0.5% (w/v) of the composition.

According to the present invention, an alpha-2 adrenergic receptor is selected from Brimonidine or a pharmaceutically acceptable salt or solvate or hydrate thereof wherein the concentration of brimonidine tartrate in the composition is from about 0.01-0.5% (w/v) of the composition.

In another embodiment, the present invention relates to compositions comprising a combination of Brinzolamide and Brimonidine or their pharmaceutical acceptable salts thereof.

In another embodiment, the present invention relates to compositions comprising a combination of Brinzolamide and Brimonidine or their pharmaceutical acceptable salts thereof along with a buffer other than borate buffer.

In another embodiment, the ophthalmic compositions of the present invention are substantially free of borate buffer and thus lacks borate-polyol complex formation and is free of microbes during storage and for the duration of its use.

In another embodiment, the ophthalmic compositions of the present invention contain Tromethamine as a sole buffer to stabilize or maintain the ophthalmic formulation at the desired pH.

In yet another embodiment, the present invention is directed to topical ophthalmic compositions for the decrease of intraocular pressure in patients with ocular hypertension or open angle glaucoma comprising a combination of Brinzolamide and Brimonidine or their pharmaceutical acceptable salts thereof optionally with pharmaceutically acceptable excipients.

In yet another embodiment, particularly the present invention is directed to develop a stable ophthalmic composition that lacks borate-polyol complex formation and is free of microbes during storage and for the duration of use.

In another embodiment, the present invention is directed to such compositions that would provide a significant improved preservation over the prior art compositions.

In a preferred embodiment, however, the ophthalmic composition is a single or multi-dose ophthalmic composition containing a combination of two or more therapeutic agents optionally with pharmaceutically acceptable excipients.

In a preferred embodiment, the therapeutic agents are selected from the groups including prostaglandin analogs (e.g., latanoprost, travoprost and unoprostone), hypotensive lipids (e.g., bimatoprost), and glucocorticoids (e.g., prednisolone, dexamethasone and lotoporednol), beta blockers such as acebutolol, atenolol, labetalol, metoprolol, propranolol, timolol (e.g., timolol maleate) and derivatives thereof, olopatadine (e.g., olopatadine hydrochloride), Carbonic anhydrase inhibitors such as brinzolamide, dorzolomide, acetazolamide, alpha-2 adrenergic agonist comprises L-norepinephrine, clonidine, brimonidine (e.g., brimonidine tartrate), emadastine, tandospirone, roscovitine, nepafenac, bradykinin, PDE4 inhibitor or combinations thereof.

In another embodiment, the compositions are typically configured for topical application to the eye (e.g., as drops directly to the eye) in a patient in need thereof.

As used herein, the term “polyol” if used, includes but not limited to, mannitol, glycerin, xylitol, sorbitol, propylene glycol or combination thereof and is present in a concentration from about 0.01% to 0.5% (w/v) of the composition.

In another embodiment, one or more pharmaceutical acceptable excipients if included would be selected from a group, but not limited to buffering agents, preservatives, tonicity agents, surfactants, viscosity-modifying agents or a suspending agent and so on.

Examples of viscosity-modifying agents or suspending agents include, without limitation, carboxyvinyl polymer (Carbomer 974 P), xanthan gum, gellan gum, sodium carboxymethyl cellulose alginic acid, carageenans. Highly preferred examples of anionic polymers include carboxyvinyl polymer, xanthan gum or a combination thereof and is present from about 0.05% to about 5.0% by weight of the composition.

A surfactant may be used, and the preferred surfactants are tyloxapol, polysorbate 80 and Polyoxyethylene (POE) (40) Hydrogenated Castor oil (or PEG (40 Hydrogenated castor oil) (HCO-40).

According to the present invention, a preservative is selected from benzododecinium halide, chlorobutanol, sodium perborate, cetrimonium chloride, thiomersal, methyl parahydroxybenzoate, propyl parahydroxybenzoate, sorbic acid and derivatives thereof, polyquaternium ammonium chloride, polyaminopropyl biguanide, phenyl mercuric nitrate, phenyl mercuric acetate, hydrogen peroxide. Instead, when the preservative is included in the present invention, the preservative is preferably benzododecinium halide, preferably benzododecinium bromide and present in the range of 0.001% to 0.1% (w/v), more preferable in the range of 0.005% to 0.03% (w/v) benzododecinium bromide.

In one of the embodiment, the preservative if present is substantially free or entirely free of any preservatives other than benzododecinium bromide (BDDB).

In one of the embodiment, the preservative if present is substantially free or entirely free of any preservatives other than Sodium perborate.

In one of the embodiment, the preservative if present is substantially free or entirely free of any preservatives other than Polyquaternium-1(PQ-1).

In another embodiments, the compositions of the present invention may include a polyol as a tonicity agent selected from the group comprising but not limited to mannitol, sorbitol or combination thereof or the like. Of these, it typically preferred that the only polyol be substantially entirely mannitol. The polyol is typically present in the range of about 0.01 w/v % to about 5 w/v % of the ophthalmic composition.

The present invention is particularly directed to the provision of multi-dose ophthalmic compositions that have sufficient antimicrobial activity to allow the compositions to satisfy the USP preservative efficacy requirements, as well as other preservative efficacy standards for aqueous pharmaceutical compositions.

In yet another embodiment various alternative preservative system were used which have better tolerability then benzalkonium chloride (BAC/BKC).

Polyquaternium-1(PQ-1) is a hydrophilic cationic polymer used as a preservative in both dry eye preparations and glaucoma medications It has been used in various USFDA approved ophthalmic products since long.

Sodium perborate, is an oxidative-type preservative which alters protein synthesis within bacterial cells through oxidative mechanisms and is effective against bacteria and the fungus Aspergillus niger. When combined with water, sodium perborate is converted to hydrogen peroxide and it has been used in several dry eye products.

In another embodiment, the compositions of the present invention in addition to therapeutic agents preferably also contains a buffering agent to stabilize or maintain the ophthalmic formulation at the desired pH. Any suitable buffering agent can be used which is compatible with the other ingredients of the ophthalmic composition of present invention. Examples of suitable ophthalmically acceptable buffering agents include but not limited to acetate buffers, citrate buffers, phosphate buffers, tromethamine and mixtures thereof. Specific buffering agents useful in the present invention include Tromethamine, sodium citrate, sodium acetate, and mixtures thereof. In the present invention, the buffer is present in at least about 0.05 w/v % to 5.0 w/v % of the ophthalmic composition.

In another embodiments, the pH adjusting agents include but not limited to, hydrochloric acid, sodium hydroxide, phosphoric acid, acetic acid and the like.

The compositions of the present invention will typically have a pH in the range of 4 to 8, preferably 5.5 to 8.0. Particularly desired pH ranges are 6.0 to 7.8 and more specifically 6.2 to 7.7. The pH of the present invention compositions are not more than 8.0.

In another embodiments, the compositions will have an osmolality of 200 to 400 or 450 milliosmoles per kilogram (mOsm/kg), more preferably 240 to 360 mOsm/kg.

In one embodiment, the present invention is directed to a multi-dose ophthalmic compositions for the decrease of intraocular pressure (IOP) in patients with ocular hypertension or open angle glaucoma comprising a combination of therapeutic agents and a method for their preparation thereof.

In another embodiment, the compositions of the present invention will generally be formulated as a sterile aqueous solutions, emulsions or suspensions so as to be compatible with the eye and/or other tissues to be treated with the compositions.

In an embodiment, the present invention provides the ophthalmic compositions in the form of aqueous liquids, solutions, emulsion, dispersion, suspension, reverse emulsion and microemulsion, nanoemulsion, nano reservoir system, in-situ gel drops, nanoparticulate system, liposomal drops, bioadhesive gel drops, drops and the like.

In another embodiment, the present invention preferably provides the ophthalmic composition for topical ophthalmic delivery comprising administering said composition in the eyes, ear, and/or nose of the humans or animals.

In yet another embodiments, any pharmaceutically acceptable packaging material may be use, preferably pharmaceutically acceptable packaging materials include but are not limited to low density polyethylene (“LDPE”), high density polyethylene (“HDPE”), polypropylene, polystyrene, polycarbonate, polyesters (such as polyethylene terephthalate and polyethylene naphthalate), nylon, polyvinyl chloride), poly(vinylidine chloride), poly(tetrafluoroethylene) and other materials known to those of ordinary skill in the art. Flexible bottles prepared from, or comprising, LDPE, HDPE or polypropylene are particularly preferred.

In an embodiment, the present invention provides a method to lower intraocular pressure in patients with glaucoma or ocular hypertension wherein the method comprises a topical application to the eye of the patient in need of an ophthalmic composition comprising a combination of Brinzolamide and Brimonidine or their salts thereof optionally with a pharmaceutically acceptable excipients.

The present invention further provides a method of using the ophthalmic compositions of present invention for lowering intraocular pressure in patients with glaucoma or ocular hypertension.

The main embodiment of the present invention is to provide an aqueous ophthalmic composition, comprising:
a therapeutically effective amount of brimonidine or their salts thereof;
a therapeutically effective amount of brinzolamide or their salts thereof;
buffers at a concentration that is at least about 0.05% w/v to 5.0% w/v of the ophthalmic composition;
a preservative and;
pharmaceutically acceptable excipients, wherein the pH of said composition is less than 8.0.

In another embodiment of the present invention, wherein the composition comprises 0.01-0.5% (w/v) of brimonidine tartrate.

In another embodiment of the present invention, wherein the composition comprises 0.01-0.5% (w/v) of brinzolamide.

In another embodiment of the present invention, wherein the buffer includes, but not limited to, acetate buffers, citrate buffers, phosphate buffers, tromethamine and mixtures thereof.

In another embodiment of the present invention, wherein the composition is substantially free of borate buffers.

In another embodiment of the present invention, wherein the pH of the composition is not more than 8.0.

In another embodiment of the present invention, wherein the composition has a pH at least 4 but less than 8.0.

In another embodiment of the present invention, wherein the preservative is selected from benzododecinium bromide (BDDB), chlorobutanol, sodium perborate, cetrimonium chloride, thiomersal, methyl parahydroxybenzoate, propyl parahydroxybenzoate, sorbic acid and derivatives thereof, polyquaternium ammonium chloride, polyquaternium-1, polyaminopropyl biguanide, phenyl mercuric nitrate, phenyl mercuric acetate, hydrogen peroxide and their mixture thereof.

In another embodiment of the present invention, wherein the composition is substantially free of benzalkonium chloride.

In another embodiment of the present invention, wherein the composition satisfies Ph. USP, Ph. Eur. B or both.

In another embodiment of the present invention, wherein one or more pharmaceutical acceptable excipients if included, would be selected from a group, but not limited to buffering agents, preservatives, tonicity agents, surfactants, viscosity-modifying agents or a suspending agent and mixtures thereof.

In another embodiment of the present invention, wherein the composition further comprising a suspending agent.

In another embodiment of the present invention, wherein the suspending agent is selected from carboxyvinyl polymer (Carbomer 974 P), xanthan gum, gellan gum, sodium carboxymethyl cellulose alginic acid, carageenans.

In another embodiment of the present invention, wherein the composition further comprising one or more polyols.

In another embodiment of the present invention, wherein the polyol if used, includes but not limited to, mannitol, glycerin, xylitol, sorbitol, propylene glycol or combination thereof.

In another embodiment of the present invention, wherein the composition is a sterile aqueous suspension.

In another embodiment of the present invention, wherein the composition is for topical ophthalmic delivery comprising administering said composition in the eyes in need thereof.

In another embodiment of the present invention, wherein the sterile aqueous suspension is suitable for ophthalmic use.

In another embodiment of the present invention, wherein the composition is administered either once a day or twice a day to each eye in need thereof.

Another embodiment of the present invention provides a method of reducing intraocular pressure in a patient in need thereof, comprising administering to the patient the composition of claims 1 to 19, the administered composition comprising a therapeutically effective amount of brimonidine tartrate and brinzolamide each at a concentration that is at least about 0.05% w/v to 5.0% w/v of the ophthalmic composition.

Another embodiment of the present invention provides use of a composition for reducing intraocular pressure in a patient in need thereof comprising safe and a therapeutically effective amount of brimonidine tartrate and brinzolamide.

Yet another embodiment of the present invention provides an aqueous ophthalmic composition, comprising:
a therapeutically effective amount of brimonidine or their salts thereof;
a therapeutically effective amount of brinzolamide or their salts thereof;
buffers at a concentration that is at least about 0.05% w/v to 5.0% w/v of the ophthalmic composition;
a preservative;
one or more polyols;
a suspending agent and;
optionally pharmaceutically acceptable excipients, wherein the pH of said composition is less than 8.0.

The scope of the present invention is illustrated by the following examples which is not meant to restrict the scope of the invention in any manner whatsoever.

The term ‘q.s.’ wherever appears in the examples is an abbreviation for ‘quantity sufficient’ which is the amount of the excipient in such quantities that is just sufficient for its use in the composition of the present invention.

### EXAMPLES

The scope of the present invention is illustrated by the following examples which is not meant to restrict the scope of the invention in any manner whatsoever.

### Example 1

| Ingredients | Quantity (% w/v) |
|---|---|
| Brinzolamide | 1.0 |
| Brimonidine Tartrate | 0.2 |
| Benzododecinium | 0.01 |
| Tromethamine (Tris Buffer) | 0.5 |
| Tyloxapol | 0.025 |
| Mannitol | 0.3 |
| Propylene Glycol | 0.75 |
| Carbomer 974 P | 0.40 |
| Sodium Chloride | 0.25 |
| Sodium Hydroxide | q.s. to adjust pH to 6.5 |
| Hydrochloric Acid | |
| Milli-Q Water | q.s. to 100 mL |

### Method of Preparation:

In one of the embodiments, there is provided a process for preparing a composition suitable for preparing Ophthalmic formulations as described herein comprising Brinzolamide and Brimonidine Tartrate Ophthalmic Suspension. The procedure is divided into four main steps:

1. Preparation of Polymer phase

2. Preparation of Brimonidine Tartrate and Buffer phase

3. Preparation of Brinzolamide and Tyloxapol slurry

4. Bulk preparation

### 1.0 Preparation of Polymer Phase:

1) Take 45% (of actual batch size) of milli Q water in a clean glass beaker.
2) Add slowly the dispensed quantity of Mannitol into the above mili-Q water under continuous stirring (rpm 800±100) to get a clear solution.
3) Slowly add dispensed quantity of Carbomer 974P into the above solution at increased rpm (2000±100) via sprinkling. Decrease the stirring rate to 1200±100 after complete addition and stirred for 60 mins. to get homogeneous dispersion.
4) Add slowly the dispensed quantity of sodium chloride to above dispersion under continuous stirring (rpm 1200±100) to get it dissolved completely.
5) Add slowly the dispensed quantity of Tromethamine (Previously dissolved in milli-Q water to prepare 10% stock solution) to above dispersion under continuous stirring (rpm 2000±100) and volume of the dispersion made up to 60% of the standard batch size and stir for 1.0 hrs to mix it completely.
6) Filter the solution through 20 μm PP filter for clarification of Polymer phase.
7) Autoclave the Carbomer phase at 121° C. for 30 min in a Schott glass bottle. Then cool it at room temperature.

### 2.0 Preparation of Brimonidine Tartrate and Buffer Phase:

1) Take 10% (of actual batch size) of milli Q water in a clean glass beaker.
2) Add slowly dispensed quantity of Propylene Glycol and Benzododecinium Bromide one by one under continuous stirring (rpm 800±100) to get a clear solution.
3) Add dispensed quantity of Brimonidine Tartrate to above solution under continuous stirring (rpm 800±100) and stir for 30 mins or till it get clear solution.
4) Volume of the solution made up to 15% (of the actual batch size) and stir for 15 mins.

### 3.0 Preparation of Brinzolamide+ Tyloxapol Slurry:

1) Take 2.0% (of actual batch size) hot milli Q water. Dissolve weighed quantity of Tyloxapol with hot milli-Q water and stir for 15 mins. Volume of the solution made upto 2.57% of the standard batch size and stir for 15 mins. Filter the solution with 0.2 μm PES filter in a separate beaker.
2) Add slowly dispensed quantity of API (Brinzolamide) to it under continuous stirring, Volume of the slurry made upto 5% (of actual batch size) and stir for 2 hrs.
3) Milled the API slurry with 2000 RPM agitator speed and 300 RPM pump flow rate for 20 mins for particle size reduction using Netzsch bead mill. After milling rinse the mill with milli Q water and volume of the slurry made upto 20% of the batch size.

### 4.0 Bulk Preparation

1) Filter the solution of Brimonidine Tartrate and Buffer phase of step 2 through 0.2μ PES filter and transferred to previously cooled autoclave phase of Carbomer 974 P of step 1 under stirring and stir for 30 mins to mix it completely.
2) Then add and mix the milled Brinzolamide and Tyloxapol slurry of step 3 to above phase under stirring and stir for 30 mins. The pH of the suspension was checked, if required adjusted to 6.5±0.3 using 1N Sodium Hydroxide/1N Hydrochloric acid solution.
3) Make up the volume with previously sterilized milli Q water upto 100.0% (of actual batch size)
4) Stir the suspension for 2 hours in aseptic conditions.
5) Fill the final suspension in previously sterilized LDPE three piece bottles, suitable for ophthalmic use.
   Conclusion of the Preservative efficacy test (PET): The present composition is formulated with 75% of label claim of Benzododecinium Bromide (BDDB) and Tromethamine Buffer and done the PET study with 75% of label claim of Benzododecinium Bromide which is complying Preservative Efficacy requirement of USP/EP and achieved sufficient anti-bacterial activity. Results are mentioned in the below tables 1 & 3.

TABLE 1
Log Reduction Value (As per USP Preservative Standards)

| | | |
|---|---|---|
| S. No. | Organism | Log Reduction |

| | | | |
|---|---|---|---|
| 1 | E. Coli | Initial Log Value | 5.27 |

| | | |
|---|---|---|
| 7 | Days | 5.27 |
| 14 | Days | 5.27 |
| 28 | Days | NI |

| | | | |
|---|---|---|---|
| 2 | S. Aureus | Initial Log Value | 5.77 |

| | | |
|---|---|---|
| 7 | Days | 5.77 |
| 14 | Days | 5.77 |
| 28 | Days | NI |

| | | | |
|---|---|---|---|
| 3 | P. Aeruginosa | Initial Log Value | 5.08 |

| | | |
|---|---|---|
| 7 | Days | 5.08 |
| 14 | Days | 5.08 |
| 28 | Days | NI |

| | | | |
|---|---|---|---|
| 4 | C. Albicans | Initial Log Value | 5.65 |

| | | |
|---|---|---|
| 7 | Days | 3.54 |
| 14 | Days | 3.81 |
| 28 | Days | NI |

| | | | |
|---|---|---|---|
| 5 | A. Brasiliensis | Initial Log Value | 5.28 |

| | | |
|---|---|---|
| 7 | Days | 3.20 |
| 14 | Days | 5.28 |
| 28 | Days | NI |

TABLE 2
The preservative efficacy standards/acceptance criteria for multi-dose
ophthalmic compositions in the U.S. is set forth in the following table:
Log Reduction

| | 7 Days | 14 Days | 28 days |
|---|---|---|---|
| Bacteria | NLT 1 log from | NLT 3 log from | NI from 14 days |
| | initial value | initial value | count at 28 days |
| Fungi | NI from initial | NI from initial | NI from initial |
| | count | count | count |

NI:—No Increase,
NA:—Not Applicable

TABLE 3
Log Reduction Value (As per EP Preservative Standards):

| | | |
|---|---|---|
| S. No. | Organism | Log Reduction |

| | | | |
|---|---|---|---|
| 1 | S. Aureus | Initial Log Value | 5.77 |

| | | |
|---|---|---|
| 24 | Hrs. | 3.77 |
| 7 | Days | 5.77 |
| 14 | Days | 5.77 |
| 28 | Days | NI |

| | | | |
|---|---|---|---|
| 2 | P. Aeruginosa | Initial Log Value | 5.08 |

| | | |
|---|---|---|
| 24 | Hrs. | 2.76 |
| 7 | Days | 5.08 |
| 14 | Days | 5.08 |
| 28 | Days | NI |

| | | | |
|---|---|---|---|
| 3 | C. Albicans | Initial Log Value | 5.65 |

| | | |
|---|---|---|
| 24 | Hrs. | NA |
| 7 | Days | 3.54 |
| 14 | Days | 3.81 |
| 28 | Days | NI |

| | | | |
|---|---|---|---|
| 4 | A. Brasiliensis | Initial Log Value | 5.28 |

| | | |
|---|---|---|
| 24 | Hrs. | NA |
| 7 | Days | 3.20 |
| 14 | Days | 5.28 |
| 28 | Days | NI |

TABLE 4
The preservative efficacy standards/acceptance criteria
for multi-dose ophthalmic compositions as per the
B-Criteria of EP is set forth in the following table:
Log Reduction#

| | 24 Hrs. | 7 Days | 14 Days | 28 days |
|---|---|---|---|---|
| Bacteria | 1 | 3 | NA | NI |
| Fungi | NA | NA | 1 | NI |

#Note:
Criteria given in terms of Log reduction in the number of viable micro-organisms against the value obtained for inoculum.
NI: No increase, NA: Not Applicable.

Further, the present invention formulation(s) is/are stable without using borate-polyol complex and complies the preservative efficacy test as per USP specification.

### Example 2

| Ingredients | Quantity (% w/v) |
|---|---|
| Brinzolamide | 1.0 |
| Brimonidine Tartrate | 0.2 |
| Benzododecinium Bromide | 0.01 |
| Tromethamine (Tris Buffer) | 0.5 |
| Tyloxapol | 0.025 |
| Mannitol | 2.3 |
| Carbomer 974 P | 0.40 |
| Sodium Chloride | 0.25 |
| Sodium Hydroxide | q.s. to adjust pH to 6.5 |
| Hydrochloric Acid | q.s. to adjust pH to 6.5 |
| Milli-Q Water | q.s. to 100 mL |

TABLE 1

| | Example 1 | Example 2 |
|---|---|---|
| Test Parameters | (US PROD-088-23) | (US PROD-88-25) |
| pH | 6.50 | 6.48 |
| Osmolality(mOsmol) | 239 | 240 |
| Viscosity(cps) CPA | 22.87 | 40.70 |
| 52Z @ 60 RPM | | |

Particle Size Distribution

| | | |
|---|---|---|
| d(0.1)μm | 0.41 | 0.45 |
| d(0.5)μm | 1.05 | 1.25 |
| d(0.9)μm | 2.49 | 2.71 |
| Zeta Potential | −33.7 | −34.1 |

It is observed from the results in Table 1 that all physiochemical properties like pH, zeta potential, viscosity, particle size of the batch manufactured with Example 1 is similar to batches manufactured in Example 2 wherein Example 1 contains a combination of two polyols and Example 2 contains single polyol. It suggests that there is no need to add two polyols in single formulation, even use of single polyol achieves the same results related to physiochemical properties. It also suggests that both these approaches can be useful for further development.

### Examples 3 & 4

| | Example 3 | Example 4 |
|---|---|---|
| Ingredients | Quantity (% w/v) | Quantity (% w/v) |
| Brinzolamide | 1.0 | 1.0 |
| Brimonidine Tartrate | 0.2 | 0.2 |
| Sodium Perborate | 0.005 | — |
| Polyquaternium-1 | — | 0.01 |
| (Polyquad) | | |
| Tromethamine | 0.5 | 0.5 |
| Tyloxapol | 0.025 | 0.025 |
| Mannitol | 0.3 | 0.3 |
| Propylene Glycol | 0.75 | 0.75 |
| Carbomer 974P | 0.40 | 0.40 |
| Sodium Chloride | 0.25 | 0.25 |
| Sodium Hydroxide | q.s. to adjust pH to 6.5 | q.s. to adjust pH to 6.5 |
| Hydrochloric Acid | q.s. to adjust pH to 6.5 | q.s. to adjust pH to 6.5 |
| Milli-Q Water | q.s. to 100 mL | q.s. to 100 mL |

TABLE 2

| | Example 3 | Example 4 |
|---|---|---|
| Test Parameters | (US PROD-088-49) | (US PROD-088-50) |
| pH | 6.54 | 6.45 |
| Osmolality(mOsmol) | 226 | 271 |
| Viscosity(cps) CPA | 62.01 | 35.58 |
| 52Z @ 60 RPM | | |

Particle Size Distribution

| | | |
|---|---|---|
| d(0.1)μm | 0.415 | 0.32 |
| d(0.5)μm | 1.05 | 0.72 |
| d(0.9)μm | 2.47 | 1.97 |
| Zeta Potential | −27.8 | −27.0 |

Results from Table 2 suggested that all physiochemical properties like pH, zeta potential, viscosity, particle size of these examples 3 & 4 are closely matching. The present invention formulations are also compared with reference product (Simbrinza) for particle size, zeta potential, rheology, drug release and found comparable in all these parameters.

### Example 5

In this example, quantity of tromethamine buffer is reduced to 0.09% from 0.5% of Example 1 and pH adjustment was done with sodium hydroxide. The reduction was done to evaluate the viscosity parameters and Sodium hydroxide may useful to increase the viscosity of this formulation. These batches further subjected to stability and characterized for various in vitro parameters.

| | Example 5a | | |
|---|---|---|---|
| | (Benzododecinium | Example 5b with | Example 5c with |
| | Bromide as a | (Polyqauternium - 1 | (Sodium Perborate |
| | preservative) | as a preservative) | as a preservative) |
| | (US-PROD-088-38) | US-PROD-088-37 | US-PROD-088-36 |
| Ingredients | Quantity (% w/v) | Quantity (% w/v) | Quantity (% w/v) |
| Brinzolamide | 1.0 | 1.0 | 1.0 |
| Brimonidine Tartrate | 0.2 | 0.2 | 0.2 |
| Benzododecinium | 0.01 | X | X |
| Bromide | | | |
| Polyquaternium-1 | X | 0.01 | X |
| Sodium Perborate | X | X | 0.005 |
| Tromethamine (Tris Buffer) | 0.09 | 0.09 | 0.09 |
| Tyloxapol | 0.025 | 0.025 | 0.025 |
| Mannitol | 0.3 | 0.3 | 0.3 |
| Propylene Glycol | 0.75 | 0.75 | 0.75 |
| Carbomer 974 P | 0.40 | 0.40 | 0.40 |
| Sodium Chloride | 0.24 | 0.24 | 0.24 |

| | |
|---|---|
| Sodium Hydroxide | q.s. to adjust pH to 6.5 |
| Hydrochloric Acid | |
| Milli-Q Water | q.s. to 100 mL |

TABLE 3
Physiochemical data of reference product (Simbrinza) batches:

| | Reference product | Reference product |
|---|---|---|
| | Lot NO 105DD | lot No 103JJ |
| | Initial | Initial |
| Description | off white | off white |
| | suspension | suspension |
| pH | 6.44 | 6.51 |
| Osmolality | 265 | 267 |
| Assay of | 100.4 | 99.5 |
| Brinzolamide | | |
| Assay of | 100.4 | 100.6 |
| Brimonidine | | |
| Particle Size | d(0.1)μm 0.320 | d(0.1)μm 0.317 |
| Distribution (μm) | d(0.5)μm 0.686 | d(0.5)μm 0.731 |
| | d(0.9)μm 1.66 | d(0.9)μm 1.62 |
| Redispersibility/ | No agglomerates were | No agglomerates were |
| Resuspendablity | observed in sample | observed in sample |
| | and on the walls of | and on the walls of |
| | container after 15 sec | container after 15 sec |
| | vigorous shaking | vigorous shaking |

TABLE 4
Stability Study results of Formulations 5a, 5b and 5c:

| | | | | |
|---|---|---|---|---|
| | | 1 Week/ | 2 Week/ | 1M (40° C./ |
| Test Parameters | Initial | 60° C. | 60° C. | NMT25% RH) |

Brinzolamide and Brimonidine Tartrate Ophthalmic
Suspension 1% & 0.2% (B. No.: US-PROD-088-38)

| | | | | |
|---|---|---|---|---|
| Description | off white | off white | off white | off white |
| | suspension | suspension | suspension | suspension |
| pH | 6.52 | 6.51 | 6.51 | 6.56 |
| Osmolality (mOsmol) | 226 | 228 | 232 | 224 |
| Viscosity(cps) | 54.42 | 46.28 | 51.09 | 45.73 |
| Assay of Brinzolamide | 98.3 | 98.8 | 99.1 | 99.0 |
| (%, By HPLC) | | | | |
| Assay of Brimonidine | 100.4 | 100.5 | 100.1 | 99.4 |
| Tartrate (%, By HPLC) | | | | |

Related Substances of Brinzolamide (%, By HPLC)

| | | | | |
|---|---|---|---|---|
| Highest Unknown | 0.03 | 0.03 | 0.03 | 0.14 |
| impurity | | | | |
| Total Impurity | 0.12 | 0.13 | 0.14 | 0.23 |

Related Substances of Brimonidine

| | | | | |
|---|---|---|---|---|
| Total Impurities | 0.04 | 0.32 | 0.61 | 0.09 |
| Particle Size | d(0.1)μm 0.311 | d(0.1)μm 0.507 | d(0.1)μm 0.453 | d(0.1)μm 0.329 |
| Distribution (in nm) | d(0.5)μm 0.731 | d(0.5)μm 1.65 | d(0.5)μm 1.41 | d(0.5)μm 0.817 |
| | d(0.9)μm 1.83 | d(0.9)μm 4.09 | d(0.9)μm; 3.54 | d(0.9)μm; 2.10 |
| Redispersibility/ | No agglomerates were | | | No agglomerates were |
| Resuspendability | observed in sample | | | observed in sample |
| | and on the walls of | | | and on the walls of |
| | container after 15 sec | | | container after 15 sec |
| | vigorous shaking | | | vigorous shaking |

Brinzolamide and Brimonidine Tartrate Ophthalmic
Suspension 1% & 0.2%, (B No.: US-PROD-088-37)

| | | | | |
|---|---|---|---|---|
| Description | off white | off white | off white | off white |
| | suspension | suspension | suspension | suspension |
| pH | 6.51 | 6.52 | 6.52 | 6.56 |
| Osmolality(mOsmol) | 226 | 227 | 229 | 224 |
| Viscosity(cps) | 57.52 | 53.10 | 51.09 | 55.58 |
| Assay of Brinzolamide | 98.1 | 98.7 | 100.7 | 99.0 |
| (%, [symbol] | | | | |
| Assay of Brimonidine | 99.6 | 100.1 | 101.7 | 99.2 |
| Tartrate (%, By [symbol] | | | | |

Related Substances of Brinzolamide (%, By HPLC)

| | | | | |
|---|---|---|---|---|
| Highest Unknown | 0.03 | 0.03 | 0.03 | 0.15 |
| impurity | | | | |
| Total Impurity | 0.16 | 0.13 | 0.14 | 0.25 |

Related Substances of Brimonidine Tartrate

| | | | | |
|---|---|---|---|---|
| Total Impurities | 0.04 | 0.33 | 0.58 | 0.10 |
| Particle Size | d(0.1)μm 0.311 | d(0.1)μm 0.447 | d(0.1)μm 0.453 | d(0.1)μm 0.329 |
| Distribution(μm) | d(0.5)μm 0.606 | d(0.5)μm 1.43 | d(0.5)μm 1.41 | d(0.5)μm 0.817 |
| | d(0.9)μm 1.40 | d(0.9)μm 3.53 | d(0.9)μm 3.54 | d(0.9)μm 2.10 |
| Redispersibility/ | No agglomerates were | NA | NA | No agglomerates were |
| Resuspendablity | observed in sample | | | observed in sample |
| | and on the walls of | | | and on the walls of |
| | container after 15 sec | | | container after 15 sec |
| | vigorous shaking | | | vigorous shaking |

Brinzolamide and Brimonidine Tartrate Ophthalmic
Suspension 1% & 0.2%, (B. No.: US-PROD-088-36)

| | | | | |
|---|---|---|---|---|
| Description | off white | off white | off white | off white |
| | suspension | suspension | suspension | suspension |
| pH | 6.61 | 6.62 | 6.60 | 6.70 |
| Osmolality(mOsmol) | 227 | 227 | 230 | 224 |
| Viscosity(cps) | 60.15 | 63.33 | 57.91 | 59.46 |
| Assay of Brinzolamide | 98.5 | 98.4 | 98.8 | 98.0 |
| (%, By HPLC) | | | | |
| Assay of Brimonidine | 99.6 | 99.7 | 99.9 | 98.0 |
| Tartrate (%, By HPLC) | | | | |

Related Substances of Brinzolamide (%, By HPLC)

| | | | | |
|---|---|---|---|---|
| Highest Unknown | 0.03 | 0.03 | 0.03 | 0.19 |
| impurity | | | | |
| Total Impurity | 0.14 | 0.13 | 0.14 | 0.33 |

Related Substances of Brimonidine Tartrate

| | | | | |
|---|---|---|---|---|
| Total Impurities | 0.05 | 0.38 | 0.64 | 0.14 |
| Particle Size | *NA | d(0.1)μm 0.507 | d(0.1)μm 0.518 | d(0.1)μm 0.369 |
| Distribution (μm) | | d(0.5)μm −1.65 | d(0.5)μm −1.68 | d(0.5)μm −0.96 |
| | | d(0.9)μm 4.09 | d(0.9)μm 3.96 | d(0.9)μm 2.23 |
| Redispersibility/ | No agglomerates were | NA | NA | No agglomerates were |
| Resuspendablity | observed in sample | | | observed in sample |
| | and on the walls of | | | and on the walls of |
| | container after 15 sec | | | container after 15 sec |
| | vigorous shaking | | | vigorous shaking |

*Not reported due to variability [symbol]
indicates data missing or illegible when filed

The stress study results depicted that the present formulations are stable and there is no drop in assay during stress study condition. There is also no increase in related substance Brinzolamide during the stress stability. The slight increase in impurity in Brimonidine tartrate was observed, however it is within the ICH impurity level. Further there is no impact on other physiochemical properties like pH, viscosity and osmolality during the stress testing. There is observed a slight increase in particle size during 1 week stress condition; however there is no increase or change in particles size from 1 week stress to 2 week stress condition, stable thereafter.

These formulations are also subjected to ICH stability study condition. Similarly, stability data at 1M 40° C./NMT25% RH indicate that all formulations are stable for pH, osmolality, viscosity, assay and impurities. There is no change in particles size during the stability in all three proposed formulation after 1 month at 40° C./NMT25% RH.

The proposed formulations using alternative preservatives to BAC have shown good stability along with compliance to preservative efficacy standard. The all proposed preservatives BDDB, PQ-1 and sodium borate are surprisingly compatible. The results indicate that the proposed formulations are surprisingly stable without using borate-polyol complex.

Also, Tromethamine (Tris) (pKa 8.1) as an alternative to boric acid buffer can be used as a substitute near neutral pH conditions. The enhanced chemical and physical stability of Tromethamine make it amenable to steam sterilization. Due to its weak basicity, it is also less corrosive to manufacturing equipments and safer to handle when compared to the inorganic bases. Tromethamine is mild base and can provide stable formulation particularly at physiological pH. The use of tromethamine buffer has imparted stability and there is no change in pH of formulations during the stress stability in all three examples 5a, 5b & 5c.

Also, surprisingly the tromethamine can alone be used for the neurotisation of carbomer as an alternative to Sodium hydroxide. It is well known that Tromethamine is an ideal neutralizer in carbomer gels that use low molecular weight alcohols as co-solvents for drug molecules. Carbomer salts of tromethamine exhibit greater compatibility with alcohols compared to the inorganic bases. Thus, along with buffering capacity it can be work as neutralization alternative to sodium hydroxide.

Re-suspendability is also a critical quality attribute and any suspension should be re-suspended similar to reference product. There is no impact on the re-suspendability of suspension of present formulations, the proposed suspension/formulations is redispersed within 15 seconds of vigorous shaking with no agglomerate and results are comparable to reference product.

Further few other parameters are also evaluated for the present invention formulations and their results, observations are described below.
(a) Zeta potential: One critical attribute to demonstrate the equivalence of present invention formulations to the reference product is zeta potential. The zeta potential is a measure of electrical charge of particles that are suspended in liquid and is a function of surface charge of the particles, any adsorbed layer at interface and nature and composition of surrounding medium. The zeta potential has proven to be extremely relevant to practical study and control of colloidal stability and flocculation processes.
The present invention stable formulations quoted in above examples were characterized for zeta potential and the results are comparable with reference products. However, slightly lower zeta of two batches could be due to testing variability.

| | Reference | Reference | US- | US- | UP- |
|---|---|---|---|---|---|
| | Lot No | Lot No | PROD- | PROD- | PROD- |
| Test | 103JJ | 105DD | 088-36 | 088-37 | 088-38 |
| Zeta | −34.5 | −30.8 | −16.7 | −16.3 | −34.7 |
| Potential | | | | | |
| (mV) | | | | | |

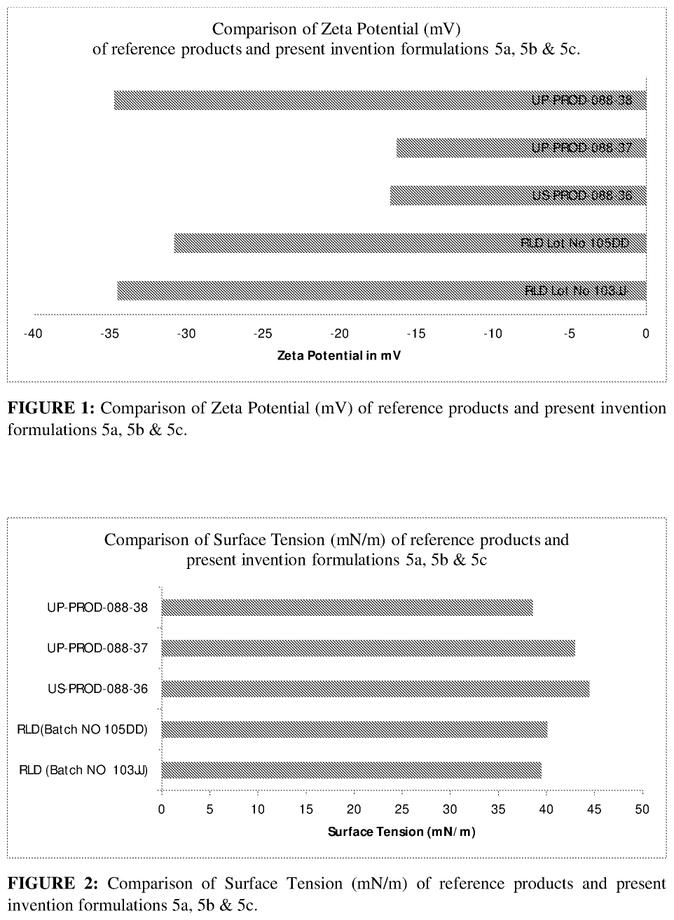

A Comparison of Zeta Potential (mV) of reference products and present invention formulations 5a, 5b & 5c are shown in FIG. 1.
(b) Surface tension: The surface tension of ophthalmic formulations affects the rate of its evaporation, the interaction with the lacrimal film of tears or the airway mucosal lining, as well as how easily it would spread along a biological surface. To minimize irritation one would expect that liquid formulations in general would mimic the natural surface tension of the particular area of administration and thus maximize interactions.
For a suspension product, the surface tension is critically important as the interfacial free energy between particles can affect the physical attributes of suspensions such as settling, aggregation, dispersability and physical stability. These attributes have the potential to affect the performance of a product, in its intended use. Furthermore, surface tension of an ophthalmic product directly influences the eye-drop volume [ref], other than the engineering design parameters of the eye-dropper drop-tip.
Surface tension of two reference product (Lot #103JJ, 105DD) and present invention formulations 5a, 5b & 5c (US-PROD-088-36, UP-PROD-088-37, UP-PROD-088-38) were measured, using a certified surface tensiometer (Wilhelmy plate method, Model DY-700 DyneMaster Surface Tensiometer, and were analyzed at Exponential Business and Technology Company (Ebatco), an ISO 17025 certified laboratory). Results suggested that surface tension of reference product and present invention formulations 5a, 5b & 5c are comparable.

| | Reference | Reference | US- | UP- | UP- |
|---|---|---|---|---|---|
| | Lot No | Lot No | PROD- | PROD- | PROD- |
| Test | 103JJ | 105DD | 088-36 | 088-37 | 088-38 |
| Surface | 39.5 | 40.1 | 44.5 | 43.0 | 38.6 |
| Tension | | | | | |
| (mN/m) | | | | | |

A comparison of Surface Tension (mN/m) of reference products and present invention formulations 5a, 5b & 5c are shown in FIG. 2.
(c) Drug release profile: The dissolution profile of a suspension-based ophthalmic eye-drop product is a critical quality attribute and a performance characteristic for a suspension. The rate of drug dissolution affects the bioavailable drug, since it is the dissolved drug fraction that is absorbed by tissues. Given that approximately 95% of each eye-drop is washed away rapidly by naso-lacrimal drainage, parameters that can affect the dissolution of a suspension are particle size, solid-state microstructure and viscosity. In terms of particle size, theory dictates that the rate of dissolution is higher for smaller particles, provided the crystals have equivalent degrees of crystallinity and solid-state microstructure. Dissolution was performed using Flow through cell (USP Type IV) Sotax Cp7 smart System (Closed System) in Simulated Tear Fluid pH 7.4. Results are presented in below table.

| | Reference | Reference | US- | UP- | UP- |
|---|---|---|---|---|---|
| % drug | Lot No | Lot No | PROD- | PROD- | PROD- |
| release | 103JJ | 105DD | 088-36 | 088-37 | 088-38 |
| 5 | 46 | 45 | 40 | 53 | 55 |
| 10 | 74 | 72 | 64 | 78 | 85 |
| 15 | 85 | 85 | 74 | 85 | 94 |
| 20 | 91 | 92 | 81 | 90 | 97 |
| 25 | 94 | 96 | 87 | 93 | 98 |
| 30 | 96 | 98 | 90 | 95 | 98 |
| 45 | 98 | 99 | 95 | 97 | 98 |
| 60 | 98 | 100 | 96 | 98 | 98 |

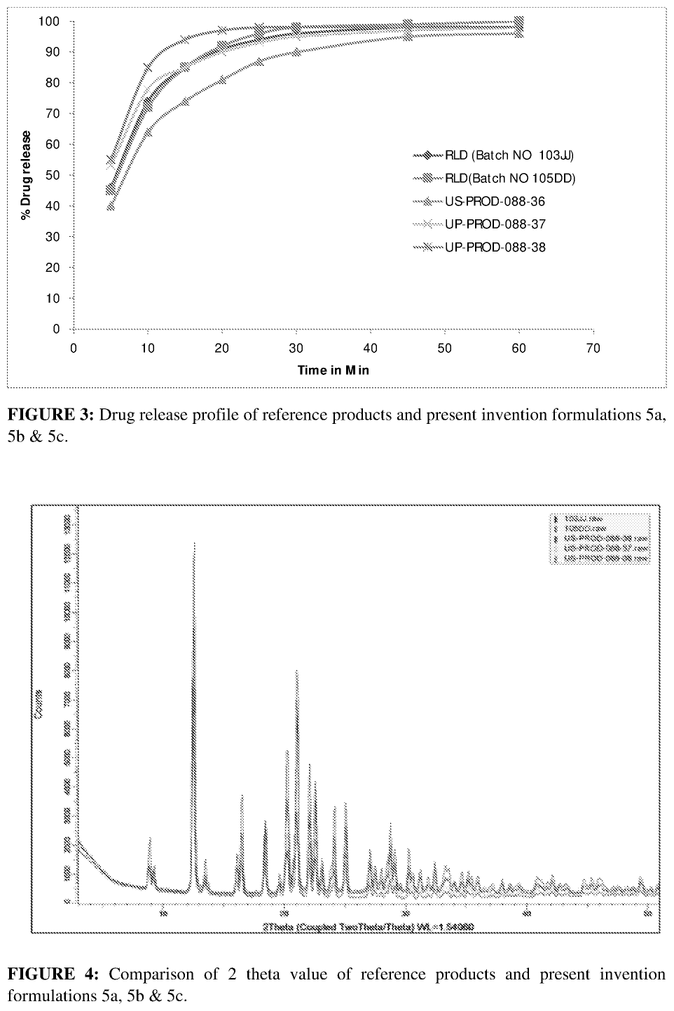

The drug release profile of reference product and present invention formulations are comparable. A drug release profile of reference products and present invention formulations 5a, 5b & 5c are shown in FIG. 3.
(d) Rheology: Viscosity of a fluid is defined as resistance to fluid flow. For an ophthalmic drug product, the residence time of the eye-drop on the ocular surface is often a function of the viscosity of the formulation. Thus, viscosity is normally considered a critical quality attribute when establishing sameness between the branded product and the ANDA product. Since viscosity is driven by the polymer in the formulation (Carbomer 974P), data from the reference product and present invention formulations as a function of increasing shear rate must be obtained to perform a complete comparative analysis.
Anionic polymers such as Carbomer 974P are shear-thinning. Eye-drops applied onto the ocular surface are subjected to shear forces imparted by blinking, thinning the applied formulation. Comparative analysis should include the rates of shear thinning of the test batches, compared to reference product. The study was done using Brookfield Ametek LV DV2T Cone and Plate viscometer with Spindle CPA-52Z at 60 RPM at 25±0.1° C.

| | Reference | Reference | US- | US- | US- |
|---|---|---|---|---|---|
| | Lot No | Lot No | PROD- | PROD- | PROD- |
| RPM | 103JJ | 105DD | 088-36 | 088-37 | 088-38 |
| 10 | 167.40 | 153.50 | 130.20 | 125.60 | 98.60 |
| 20 | 118.60 | 113.90 | 93.02 | 89.76 | 74.88 |
| 30 | 97.36 | 94.88 | 76.59 | 73.17 | 65.42 |
| 40 | 84.41 | 82.79 | 66.51 | 63.72 | 59.53 |
| 50 | 75.16 | 74.42 | 59.35 | 56.74 | 55.07 |
| 60 | 68.21 | 68.06 | 50.23 | 51.94 | 51.47 |
| 70 | 62.72 | 62.99 | 47.09 | 48.10 | 48.50 |
| 80 | 58.49 | 58.60 | 44.55 | 45.11 | 46.28 |
| 90 | 54.88 | 55.09 | 44.55 | 42.58 | 44.03 |
| 100 | 51.90 | 52.09 | 42.32 | 40.56 | 42.14 |

The results indicate that the rheology of reference product and present invention formulations are comparable.
(e) XRD Study: The crystallinity is a critical parameter for suspension characterization, and it impact on drug dissolution and drug release into the eyes. Any difference in crystallinity/polymorph will impact on to the bioavailability of the drug in eye. XRD study was done to characterize the polymorph/crystalline nature in drug product. Bruker AXX/DS focus using Lynx eye detector was used for XRD determination.
An overlay of the XRPD patterns of the crystals harvested from the batches of reference product and present invention formulations appears in FIG. 4. The five XRPD patterns display a number of discrete and well-resolved diffraction peaks, indicating that the samples are crystalline. The patterns also display peaks at very similar °2θ positions, as shown in the stacked plot in FIG. 4, suggesting that they contain the same solid form or mixture of solid forms, i.e., the same polymorph.

| | 2 theta value | | | | |
|---|---|---|---|---|---|
| | Reference | Reference | US- | US- | US- |
| | Lot No | Lot No | PROD- | PROD- | PROD- |
| S. No | 103JJ | 105DD | 088-36 | 088-37 | 088-38 |
| 1 | 12.551 | 12.50 | 12.47 | 12.49 | 12.50 |
| 2 | 16.510 | 16.489 | 16.482 | 16.500 | 16.491 |
| 3 | 18.432 | 18.42 | 18.35 | 18.458 | 18.421 |
| 4 | 20.24 | 20.20 | 20.21 | 20.23 | 20.224 |
| 5 | 21.037 | 21.03 | 21.02 | 21.068 | 21.028 |
| 6 | 22.094 | 22.09 | 22.041 | 22.098 | 22.093 |
| 7 | 22.556 | 22.52 | 22.539 | 22.566 | 22.547 |
| 8 | 23.121 | 23.10 | 23.059 | 23.11 | 23.093 |
| 9 | 24.135 | 24.127 | 24.077 | 24.134 | 24.115 |
| 10 | 25.088 | 25.05 | 24.949 | 25.071 | 25.089 |

A comparison of 2 theta value of reference products and present invention formulations 5a, 5b & 5c are shown in FIG. 4.

### Utility of the Present Invention

The present inventors have provided an aqueous ophthalmic composition. The composition contains either Benzododecinium Bromide (BDDB) or Sodium Perborate or Polyquaternium-1 as a preservative for improved preservation of the compositions. The composition has shown potential effects in lowering intraocular pressure in patients with glaucoma or ocular hypertension.

1. An aqueous ophthalmic composition, comprising:
a therapeutically effective amount of brimonidine or their salts thereof;
a therapeutically effective amount of brinzolamide or their salts thereof;
buffers at a concentration that is at least about 0.05% w/v to 5.0% w/v of the ophthalmic composition;
a preservative and;
pharmaceutically acceptable excipients, wherein the pH of said composition is less than 8.0.

2. The composition as claimed in claim 1, wherein the composition comprises 0.01-0.5% (w/v) of brimonidine tartrate.

3. The composition as claimed in claim 1, wherein the composition comprises 0.01-0.5% (w/v) of brinzolamide.

4. The composition as claimed in claim 1, wherein the buffer includes, but not limited to, acetate buffers, citrate buffers, phosphate buffers, tromethamine and mixtures thereof.

5. The composition as claimed in claim 1, wherein the composition is substantially free of borate buffers.

6. The composition as claimed in claim 1, wherein the pH of the composition is not more than 8.0.

7. The composition as claimed in claim 1, wherein the composition has a pH at least 4 but less than 8.0.

8. The composition as claimed in claim 1, wherein the preservative is selected from benzododecinium bromide (BDDB), chlorobutanol, sodium perborate, cetrimonium chloride, thiomersal, methyl parahydroxybenzoate, propyl parahydroxybenzoate, sorbic acid and derivatives thereof, polyquaternium ammonium chloride, polyquaternium-1, polyaminopropyl biguanide, phenyl mercuric nitrate, phenyl mercuric acetate, hydrogen peroxide and their mixture thereof.

9. The composition as claimed in claim 1, wherein the composition is substantially free of benzalkonium chloride.

10. The composition as claimed in claim 1, wherein the composition satisfies Ph. USP, Ph. Eur. B or both.

11. The composition as claimed in claim 1, wherein one or more pharmaceutical acceptable excipients if included, would be selected from a group, but not limited to buffering agents, preservatives, tonicity agents, surfactants, viscosity-modifying agents or a suspending agent and mixtures thereof.

12. The composition as claimed in claim 1, wherein the composition further comprising a suspending agent.

13. The composition as claimed in claim 12 wherein the suspending agent is selected from carboxyvinyl polymer (Carbomer 974 P), xanthan gum, gellan gum, sodium carboxymethyl cellulose alginic acid, carageenans.

14. The composition as claimed in claim 1, wherein the composition further comprising one or more polyols.

15. The composition as claimed in claim 14 wherein the polyol if used, includes but not limited to, mannitol, glycerin, xylitol, sorbitol, propylene glycol or combination thereof.

16. The composition as claimed in claim 1, wherein the composition is a sterile aqueous suspension.

17. The composition as claimed in claim 1, wherein the composition is for topical ophthalmic delivery comprising administering said composition in the eyes in need thereof.

18. The composition as claimed in claim 1, wherein the sterile aqueous suspension is suitable for ophthalmic use.

19. The composition as claimed in claim 1, wherein the composition is administered either once a day or twice a day to each eye in need thereof.

20. A method of reducing intraocular pressure in a patient in need thereof, comprising administering to the patient the composition of claim 1, the administered composition comprising a therapeutically effective amount of brimonidine tartrate and brinzolamide each at a concentration that is at least about 0.05% w/v to 5.0% w/v of the ophthalmic composition.

21. (canceled)

22. An aqueous ophthalmic composition, comprising:
a therapeutically effective amount of brimonidine or their salts thereof;
a therapeutically effective amount of brinzolamide or their salts thereof;
buffers at a concentration that is at least about 0.05% w/v to 5.0% w/v of the ophthalmic composition;
a preservative;
one or more polyols;
a suspending agent and;
optionally pharmaceutically acceptable excipients, wherein the pH of said composition is less than 8.0.
